(19) United States
(12) Patent Application Publication
(10) Pub. No.: US 2014/0352712 A1
(43) Pub. Date: Dec. 4, 2014

(54) Compositions And Methods For Permanent Shaping Of Hair

(71) Applicants: Geoffrey Hawkins, Yardley, PA (US); Vasile Ionita-Manzatu, Bethpage, NY (US); Barbara Anne Wolf, Scarsdale, NY (US); Lavinia C. Popescu, Jackson Heights, NY (US); Jack Lombardi, Massapequa, NY (US)

(72) Inventors: Geoffrey Hawkins, Yardley, PA (US); Vasile Ionita-Manzatu, Bethpage, NY (US); Barbara Anne Wolf, Scarsdale, NY (US); Lavinia C. Popescu, Jackson Heights, NY (US); Jack Lombardi, Massapequa, NY (US)

(21) Appl. No.: 13/908,059

(22) Filed: Jun. 3, 2013

(51) Int. Cl. A61K 8/26; A61Q 5/04; A45D 7/06

(52) U.S. Cl. CPC A61K 8/26; A45D 7/06; A61Q 5/04; A61K 2800/40
USPC 132/206

(57) ABSTRACT

A hair shaping topical composition comprising materials that emit electromagnetic radiation at wavelengths that affect tertiary structure (breaking of disulfide) bonds in human hair, and that bring about changes in secondary structure of hair proteins. The intensity of the radiation is controlled and sufficient to cause or facilitate altering of protein structure. The invention includes methods of using such topical compositions. Testing indicates that the hair reshaping is permanent and there is no damage to hair of the type characteristic of chemical treatments.

This application is a divisional of U.S. Ser. No. 13/201,043, which is a national stage filing of PCT/US2010/24641 filed Feb. 19, 2010, which claims priority of U.S. 61/153,828, filed Feb. 19, 2009, herein incorporated by reference, in its entirety.

### FIELD OF THE INVENTION

The invention is in the field of hair styling. More particularly, it is in the field of permanent hair shaping by non-chemical means.

### BACKGROUND OF THE INVENTION

### Human Hair

U.S. Pat. No. 5,395,490 is herein incorporated by reference, in its entirety. FIGS. 1, 2A, 2B, 4A and 4B in U.S. Pat. No. 5,395,490 diagram the structure of human hair fibers, the protein components of hair, and energy levels of the disulfide bond.

A fiber of human hair comprises three main morphological components: the cuticle, the cortex, and the cell membrane complex, which itself is comprised of a protein matrix of keratin peptide chains, such as cysteine. A medulla may also be present. These peptide chains are linked to each other by disulfide bonds. The natural shape and structural integrity of human hair fiber depend, in part, on the orientation of the disulfide bonds which link the protein chains. They also depend on the secondary structure of the keratin fibers. However, it is generally thought that alteration of the disulfide bonds is necessary and/or useful to effect long term changes in the shape of hair, so that treatments that do not rearrange the disulfide bonds may result only in temporary changes in hair shape or are less effective at long term reshaping of hair. For example, the use of heat and moisture to style hair may create temporary waving of the hair. However, the styled hair will return to its natural shape after a short time, as a result of exposure to moisture in the air or washing. The use of heat and moisture to style hair may break and reconfigure hydrogen bonds in the hair, but the disulfide bonds are not substantially affected. It is thought that hydrogen bonds, by themselves, are insufficient to hold the shape of hair for a significant time, because the stronger disulfide bonds eventually force the hair to reassume its original shape. Thus, a permanent change in the shape of hair is thought to involve the cleaving and reforming a substantial number of disulfide bonds, but alteration of other tertiary structure and alteration of secondary structure is not ruled out.

### Chemical Treatments

Hair styling or shaping (i.e. straightening and curling) by treating the hair with chemical agents is well known. These include treatments that use reagents to break and reassemble the disulfide bonds that link protein molecules in the hair. Such reagents include mercaptans, alkali, aldehydes, etc. These styling methods are multi-step, time consuming and relatively expensive processes. Existing products also produce mal odor which is not desirable. First, the disulfide bonds are reduced with a sulfur agent, mechanical stress is applied to rearrange the disulfide bonds, then new disulfide bonds are constituted by applying an oxidizing agent (i.e. an alkali). Furthermore, chemical treatments, while effective, are considered harsh and damaging to human hair and skin. Some negative effects of hair styling include dry, brittle or limp hair; a loss of shine and/or color; damage to the scalp skin and damage to protein bonds in the hair other than the disulfide bonds. Damage to lipids in the exocuticle, swelling of the hair fiber and lifting of the cuticle also occur. Furthermore, chemical treatments are topically applied in a broad way, meaning that it is difficult to control the number or location of the affected disulfide bonds. Also, once the reducing agent is topically applied, the reducing reaction cannot be turned off immediately. Time is needed to apply the oxidizing agent and stop the process.

### Treatments Involving Light

The use of electromagnetic radiation to change the shape of human hair is also known.

There are techniques that use light to directly affect the disulfide bonds that link protein molecules in the hair, and there are techniques that use light as an adjunct to other manipulations of the disulfide bonds (i.e. to accelerate one or more chemical process). Thus, “directly affect” or “direct effect” mean that a substance emits electromagnetic radiation that is absorbed by and that excites disulfide bonds, without first being absorbed by some other material.

U.S. Pat. No. 5,395,490 discloses a method of reshaping human hair by using electromagnetic radiation to rearrange disulfide bonds within the hair. During the time that the hair is exposed to the electromagnetic energy, stress is applied to the hair. As a result, once the disulfide bond is broken, each S atom is available to form a different bond with some other dissociated S atom. The structure of the new bond is determined in part by the stress.

The energy required to raise an isolated disulfide bond from its ground state to the continuum (i.e. the dissociation energy) is reportedly about 2.2 eV. For a given bond that is raised to the continuum (i.e. the bond is cleaved), this energy may be supplied from a single photon or from a series of photons.

There is a range of photon frequencies that may be used to cleave the disulfide bonds, however, the most efficient process takes advantage of a resonance condition. The '490 reference suggests that the energy levels of an isolated S₂ molecule lie within a frequency range of 2×10¹³ to 1×10¹⁵ Hz (corresponding to about 0.30 to 15 μm wavelength or about 0.08 to 4.13 eV). However, the '490 reference suggests that in hair, the disulfide bond is subject to other forces, and therefore a frequency range of 1×10¹³ to 2×10¹⁵ Hz (corresponding to about 0.15 to 30 μm wavelength or 0.04 to 8.3 eV) is preferred. By bombarding hair with photons in this range of resonant frequencies for a length of time, the disulfide bonds will move between their natural energy states (or modes of vibration), with some bonds being excited to the continuum state.

Nevertheless, U.S. Pat. No. 5,395,490 fails to disclose a composition that comprises a material that is able to radiate in a wavelength range around 20 μm. It fails to disclose applying the composition to the hair. It fails to disclose activating the material in the composition to radiate in a wavelength range around 20 μm. It fails to disclose methods of treating the hair, as disclosed herein. Furthermore, the '490 patent applies radiation to the disulfide bonds from complicated high and low frequency wave form generators and supporting electronics. In fact, the present invention suggests a device no more complicated than a hair dryer. Also, '490 discloses a range of photon energies 0.04 to 8.3 eV, that includes the dissociation energy of S₂, about 2.2 eV. This is unlike the present invention where a device capable of producing photons at 2.2 eV is neither required, nor preferred.

WO/1994/010873 and WO/1994/010874 disclose methods of treating hair, in particular human head hair, for cosmetic purposes. The hair is exposed to light with an intensity and wavelength chosen so that the protein structure of the hair is altered to produce the desired cosmetic effect.

In WO/1994/010873 the effect is shaping hair. However, the reference discloses using light of wavelength 400 to 600 nm (0.4-0.6 μm), well below the approximately 20 nm described in the present invention. A single photon having wavelength of 400 to 600 nm “carries” about 2.05-3.0 eV of energy (which lies within the 0.04 to 8.3 eV range of the '490 patent, above). As noted, the energy required to raise a disulfide bond from its ground state to the continuum is, reportedly, about 2.2 eV. Thus, the '873 reference suggests using a narrower range of frequencies than the '490 patent, but centered around the S₂ dissociation energy. It is reasonable to expect that a wider range of frequencies disclosed in the '490 patent will be more efficient at cleaving disulfide bonds than the narrow range of frequencies disclosed in the '873 reference.

In WO/1994/010874 the cosmetic effect in view, is improved hair coloring. In particular, for the support of the chemical coloring of head hairs, light is used having a wavelength between approximately 600 nm and 1200 nm, so that a change of enzyme coordinate and/or a change of the redox potentials results. It is reported that hair coloring is improved, i.e. the colors are more brilliant than without influence of light, and less colorant is necessary than with conventional coloring. 600 to 1200 nm (0.6-1.2 μm) is well below the approximately 20 μm utilized in the present invention.

Furthermore, WO/1994/010873 and WO/1994/010874 fail to disclose a composition that comprises a material that is able to radiate in a wavelength range around 20 μm. They fail to disclose applying such a composition to the hair. They fail to disclose activating the material in the composition to radiate in a wavelength range around 20 μm. They fail to disclose methods of treating the hair, as disclosed herein. In '873 and '874, electromagnetic energy is supplied by a device; an argon laser, for example. This is unlike the present invention where a device capable of producing photons at 2.2 eV is neither required, nor preferred. Furthermore, the present invention does not require lasers and the supporting electronics to apply radiation to the disulfide bonds, as described in these patents. In fact, the present invention suggests a device no more complicated than a hair dryer.

U.S. Pat. No. 5,858,179 discloses a combination of chemicals and electromagnetic radiation used to alter the physical characteristics of keratinic fibers such as mammalian or human hair. A non-irritating, non-reactive disulfide, in the form of a solution or gel, is first contacted with the hair. Electromagnetic radiation is then applied to the hair to photo-chemically convert the disulfide into a dithiol. The dithiol breaks the disulfide bonds in the hair, so that the hair can be permanently reshaped.

U.S. Pat. No. 5,858,179 fails to disclose a composition that comprises a material that is able to radiate in a wavelength range around 20 μm. It fails to disclose applying such a composition to the hair. It fails to disclose activating the material in the composition to radiate in a wavelength range around 20 μm. It fails to disclose methods of treating the hair, as disclosed herein. U.S. Pat. No. 5,858,179 does not use electromagnetic radiation directly on the disulfide bond, to break the bond. Rather, the radiation used is chosen to convert free disulfide into dithiol using a reported wavelength of 200 to 530 nm (2.3 to 6.2 eV). Furthermore, the present invention does not require a device to generate electromagnetic radiation at specific frequencies. Rather, the present invention suggests a device no more complicated than a hair dryer.

U.S. Pat. No. 3,863,653 discloses a method and apparatus for treating fibers by enclosing them within a resonant cavity to which high frequency current is supplied, the resonant frequency and impedance of said cavity being matched to that of its supply. This method is really an adjunct to a chemical treatment method. U.S. Pat. No. 3,863,653 uses high frequency radiation to heat hair from the inside, thereby accelerating the chemical reactions and reducing the time that the hair must be exposed to the potentially damaging chemicals. The frequency of radiation disclosed is from 10-4000 MHz, wholly unsuitable for use in the present invention.

### Tourmaline

Tourmaline is an acentric rhombohedral borosilicate characterized by six-membered tetrahedral rings. It is a semi-precious stone, and a crystal silicate compounded with varying amount of elements such as aluminium, iron, magnesium, sodium, lithium, or potassium.

The compositions of tourmaline vary widely, and one general formula has been written as

XY₃Z₆(T₆O₁₈)(BO₃)₃V₃W,

where, X═Ca, Na, K, vacancy; Y═Li, Mg, Fe²⁺, Mn²⁺, Zn, Al, Cr³⁺, V³⁺, Fe³⁺, Ti⁴⁺; Z═Mg, Al, Fe³⁺, Cr³⁺, V³⁺; T=Si, Al, B; B═B, vacancy; V═OH, O; W═OH, F, O (Hawthorne and Henry 1999, Classification of the minerals of the tourmaline group. European Journal of Mineralogy, 11, 201-215).

Fourteen end-members are recognized by the International Mineralogical Association (IMA) and Hawthorne and Henry (1999) have grouped these into three principal groups, based on the dominant occupancy of the X site. These groups are the alkali group, the calcic group and the X-site vacant group. The following table with updated information is reproduced from http://www.geol.lsu.edu/henry/Research/tourmaline/TourmalineClassification.htm.

| | | | | | | | |
|---|---|---|---|---|---|---|---|
| Species | (X) | (Y₃) | (Z₆) | T₆O₁₈ | (BO₃)₃ | V₃ | W |

Alkali tourmalines

| | | | | | | | |
|---|---|---|---|---|---|---|---|
| Elbaite | Na | Li_{1.5} Al_{1.5} | Al₆ | Si₆O₁₈ | (BO₃)₃ | (OH)₃ | (OH) |
| Schorl | Na | Fe²⁺₃ | Al₆ | Si₆O₁₈ | (BO₃)₃ | (OH)₃ | (OH) |
| Dravite | Na | Mg₃ | Al₆ | Si₆O₁₈ | (BO₃)₃ | (OH)₃ | (OH) |
| Olenite | Na | Al₃ | Al₆ | Si₆O₁₈ | (BO₃)₃ | (O)₃ | (OH) |
| Chromdravite | Na | Mg₃ | Cr₆ | Si₆O₁₈ | (BO₃)₃ | (OH)₃ | (OH) |
| Buergerite | Na | Fe³⁺₃ | Al₆ | Si₆O₁₈ | (BO₃)₃ | (O)₃ | F |
| Povondraite | Na | Fe³⁺₃ | Fe³⁺₄Mg₂ | Si₆O₁₈ | (BO₃)₃ | (OH)₃ | O |
| Vanadiumdravite | Na | Mg₃ | V₆ | Si₆O₁₈ | (BO₃)₃ | (OH)₃ | (OH) |

Calcic tourmalines

| | | | | | | | |
|---|---|---|---|---|---|---|---|
| Liddicoatite | Ca | Li2Al | Al₆ | Si₆O₁₈ | (BO₃)₃ | (OH)₃ | F |
| Uvite | Ca | Mg₃ | MgAl₅ | Si₆O₁₈ | (BO₃)₃ | (OH)₃ | F |
| Hydroxy- | Ca | Fe²⁺₃ | MgAl₅ | Si₆O₁₈ | (BO₃)₃ | (OH)₃ | (OH) |
| feruvite | | | | | | | |

X-site vacant tourmalines

| | | | | | | | |
|---|---|---|---|---|---|---|---|
| Rossmanite | — | LiAl₂ | Al₆ | Si₆O₁₈ | (BO₃)₃ | (OH)₃ | (OH) |
| Foitite | — | Fe²⁺₂Al | Al₆ | Si₆O₁₈ | (BO₃)₃ | (OH)₃ | (OH) |
| Magnesiofoitite | — | Mg₂Al | Al₆ | Si₆O₁₈ | (BO₃)₃ | (OH)₃ | (OH) |

Hawthorne and Henry (1999) also postulate at least 27 other tourmalines that have yet to be verified. Thus, in speaking of tourmaline, there are substantial differences (as well as similarities) among varieties. Some reported properties of tourmalines include: specific gravity: 2.96-3.31; index of refraction: 1.610-1.735; birefringence: 0.016-0.080; pleochroism: strong in all species; hardness: 7.0-7.5.

In terms of the present invention, performance may vary from one variety to another. In particular, emissivity and absorption spectra may vary from one variety to another. Also, the intensity of emitted radiation and the activation energy may vary from one variety to another. When used in particulate form in compositions of the present invention, these properties of tourmaline will also depend on the particle size and the concentration.

### Tourmaline-Containing Products

The use of tourmaline in hair products is known. For example, a product called IB Shield Humidity Lock-Out Shine Spray by Jonathan Product describes its use of tourmaline by saying “Tourmaline & Amethyst: Charged ionic crystal blend known to improve shine, smoothness, and manageability of hair.” Further description includes “Charged ions & Far Infrared energy help revitalize the scalp to maintain optimum hair health.”

Hai Flat Iron Fluid by Angles BeautyCare Group contains tourmaline, which the manufacturer asserts, “is claimed to deliver weightless moisture and increased absorption for beautifully conditioned hair, protect it against heat damage, reduce static, and provide longer lasting color and gorgeous shine.”

Nothing in the descriptions of these products suggests a composition that comprises a tourmaline (or any other material) that is able to radiate in a wavelength range around 20 μm, and nothing suggests activating such a material to radiate in a wavelength range around 20 μm. Even if the tourmaline does radiate in this range, nothing in the prior art suggests that the intensity is sufficient to achieve a significant permanent reshaping of human head hair. To the best of the applicants' knowledge, in these products, as well as others, tourmaline is not reported to provide hair reshaping benefits. Hair reshaping is never mentioned.

Tourmaline hair dryers are also known. Such hair dryers contain tourmaline crystals that deliver negative ions and far-infrared heat, which, reportedly, dries hair from the inside out. As a result, a person can dry hair faster, and the hair is left healthy and shiny with optimum manageability. Flat irons for shaping hair are also known to contain tourmaline. Typically, it is reported that the tourmaline supplies negative ions that yield softer and shinier hair, while infrared heat is associated with improved hair moisture and luster. Hair brushes and hair setting rollers with tourmaline are known. Often, the benefit associated with tourmaline is less frizz, due to an ionic effect. None of these appliances, suggests a composition that comprises a material that is able to radiate in a wavelength range around 20 μm, or at an intensity that is sufficient to achieve a significant permanent reshaping of human head hair. They fail to disclose applying such a composition to the hair. They fail to disclose activating the material in the composition to radiate in a wavelength range around 20 μm. They fail to disclose methods of treating the hair, as disclosed herein.

### SUMMARY OF THE INVENTION

The present invention is a hair shaping topical composition comprising one or more materials that emit or are induced to emit electromagnetic radiation at wavelengths that directly affect the disulfide (S—S) bonds in hair (a form of tertiary protein structure). As we will see, however, the compositions and methods of the invention also affect secondary structure of hair proteins. The photon energies employed are well below the dissociation energy of a ground state disulfide bond. The intensity of the radiation is controlled and sufficient to break and/or weaken enough disulfide bonds, so that hair reshaping is enabled or facilitated. The treatment may be effective on its own or as an adjunct.

Such techniques are non-chemical. By “non-chemical” we mean that there is no need for a user to apply a molecule that acts as a reagent or catalyst in respect of disulphide bond cleavage and reformation. By “non-chemical” we further mean that pure energy is supplied to the disulfide bonds in hair. In this way, we may avoid all or most of the damage associated with chemical treatments. By supplying a radiation that is specific to and resonant with the disulfide bond, collateral damage is minimized. Also, as soon as the radiation is removed, the process of bond cleavage stops, unlike mixtures of reagents which cannot be separated, and the reaction runs until one or more reagents are exhausted.

The invention includes compositions that may be washed out of the hair after reshaping has occurred, and compositions that are intended to remain in the hair for additional or extended benefits. The invention includes methods of using a topical composition that comprises one or more materials that emit or are induced to emit electromagnetic radiation at wavelengths that affect the S—S bonds in hair. Testing indicates that the hair reshaping is permanent and there is no damage to hair of the type characteristic of chemical treatments.

### DESCRIPTION OF THE FIGURES

FIG. 1 is a graph of the emissivity vs. wavelength of red tourmaline at 78° C.

FIG. 2 is a graph of the radiance vs. wavelength of red tourmaline at 78° C.

FIG. 3 is depicts the time need for various amounts of red tourmaline to dissociate a given number of disulfide bonds.

FIGS. 4a-4h show samples of curly hair straightened with a composition of the present invention, and various control samples.

FIG. 5 shows a curly control sample along side two samples of hair straightened with a composition of the present invention.

FIG. 6 shows the effect of hair treatments on the break strength of human hair

FIG. 7 shows the effectiveness of various hair treatments for straightening human hair.

FIG. 8 shows the results of a colorimetric disulfide bond analysis, for samples of hair treated in various ways.

### DETAILED DESCRIPTION OF THE INVENTION

The present invention lies in the unexpected discovery that a permanent reshaping of human head hair, via non-chemical disulfide bond reorganization, can be achieved with electromagnetic energy that is supplied by a topical composition. In the present context, “topical” means applied to the surface of the hair, particularly human head hair. The electromagnetic energy directly affects or excites the disulfide bond, as opposed to be being used as an adjunct to other manipulations of the disulfide bonds, as in U.S. Pat. No. 5,858,179 and U.S. Pat. No. 3,863,653, and described above.

The present invention also lies in the surprising discovery that certain materials can be incorporated into stable, commercially acceptable, topical hair products in quantities that are sufficient to directly affect a permanent reshaping of human head hair, despite the fact that the emitted photon energy is well below the dissociation energy of a ground state disulfide bond.

Throughout the specification, the word “permanent” in reference to hair reshaping treatments, means that the shape of the hair is maintained until the hair grows out. Preferably, if the treated hair is exposed only to ambient atmospheric conditions, the new shape is maintained for at least one week, more preferably, at least one month and most preferably, at least two months. Furthermore, if treated hair is saturated (i.e. during bathing), the new shape may be lost, however, “permanent” also means that, once dried, saturated hair will revert to its post treatment shape, to a substantial degree.

Throughout the specification, “comprising” means that a collection of objects is not necessarily limited to those recited.

### Criteria for Suitable Materials and Compositions

Asking a commercially acceptable personal care composition to supply sufficient energy for reshaping human hair, while remaining reasonably priced and meeting aesthetic and regulatory requirements, places a long list of requirements on the composition. It is surprising that the criteria discussed herein, could be met successfully.

a. Wavelength, Intensity, Temperature

To achieve “permanent” reshaping of the hair, the electromagnetic radiation provided must be of the right wavelengths and must be of sufficient intensity. If the wavelengths do not correspond to the differences in energy levels of the disulfide bonds, then the bonds will not be excited. Furthermore, if the intensity is too low, then too few disulfide bonds will make it to the continuum state, because an excited bond will fall back to a lower energy level before absorbing another photon.

For any given material that we might consider in a personal care product, temperature is the most important factor affecting both wavelength and intensity. To a large extent, the temperature of a material determines the intensity and wavelength distribution of radiation emitted by the material. Compositions of the present invention will generally be exposed to temperatures between about 25° C. and 175° C. Therefore, a suitable material is one that, between about 25° C. and 175° C., emits electromagnetic radiation in a range of wavelengths that are able to excite disulfide bonds within the environment of human hair, and at an intensity that is useful to reshape human hair.

Concerning wavelength, the wavelengths emitted by the suitable material must correspond to the differences in energy levels of the disulfide bonds in human hair. U.S. Pat. No. 5,395,490 suggests that a range of wavelengths of about 0.15 to 30 μm, is preferred. While this range of wavelengths is known to be useful to cleave disulfide bonds (tertiary structure), we have observed changes in hair secondary structure, as well. 0.15 to 30 μm covers most of the near and middle infrared. Depending on the classification, and there are several, this range may also cover a small portion (about 3%) of the far infrared, which extends to about 1000 μm wavelengths. On the other hand, some classifications suggest that the middle infrared extends up to 40 μm. The point is, that at the time of the present invention, “the boundaries between the near, mid and far-infrared regions are not agreed upon and can vary” (see “CoolCosmos Infrared Astronomy Tutorial: Near, Mid, and Far Infrared”—http://coolcosmos.ipac.caltech.edu/cosmic_classroom/ir_tutorial/irregions.html).

As note above, a suitable material of the present invention is one that, between about 25° C. and 175° C., emits electromagnetic radiation at an intensity that is useful to reshape human hair. An intensity is considered “useful to reshape human hair” if the hair being treated can be reshaped in a commercially acceptable amount of time. By “commercially acceptable amount of time” we mean less than about one hour, more preferably, less than about 30 minutes, more preferably still, less than about 10 minutes, most preferably less than about 5 minutes. This time to reshape hair is measured as follows. After a composition according to the present invention is applied to a section of hair, the time to reshape the section of hair is measured from the moment the section of hair is placed in tension, until the tension is released.

So, if an otherwise useful material would require an unacceptably long time to effect the desired change (3 hours, for example), then that material is less suitable or not at all suitable for use in the present invention, because such a product has low commercial viability. On the other hand, greater intensity means more photons which means more disulfide bonds are being broken and being broken faster. In general, the greater the intensity of the applied radiation, the more dramatic the effect of hair reshaping and/or the effect is achieved in a shorter amount of time.

Now, intensity (or better, radiance) of a material, is the energy per second emitted from a unit area of the material, into a unit solid angle. Radiance depends on the temperature of the material. Thus, to find a suitable material, one could begin by looking at radiance verses wavelength curves of various materials, to find those materials that have a more prominent intensity in the 0.15 to 30 μm wavelength range, when heated to the temperature range of interest, i.e. 25° C.-175° C. or 40° C.-60° C. or 60° C.-80° C. and so forth. Determining what is a useful intensity may best be done by trial and error. A candidate material may be incorporated into a base hair composition, and applied to the hair in commercially reasonable amounts. If the hair can be effectively styled in a commercially acceptable amount of time, then the intensity may be considered useful.

Aside from wavelength and intensity, other parameters should be considered when attempting to identify a suitable material according to the present invention.

b. Emissivity

In U.S. Pat. No. 5,395,490 and other prior art, the source of radiation is a sophisticated electronic, multi-frequency electromagnetic wave generating device, that has its own power source. By design, most of the supplied power is converted into electromagnetic energy and the intensity of radiation at a given wavelength can be controlled to arbitrarily high precision. This is very different from the present invention, wherein the power source is the heat supplied to the suitable material (as from a hair dryer or flat iron), which is re-radiated in a wavelength-intensity spectrum that is characteristic of the material. The input power is limited to what is safely supplied by a generic consumer hair dryer or flat iron. Thus, not having an essentially unlimited supply of power, it is important that a suitable material be relatively efficient at re-radiating the energy that it absorbs so that the intensity will be useful. Thus, in addition to looking at the radiance of a potential suitable material, one should also look at the emissivity (a measure of a material's ability to radiate the energy that the material has absorbed). An example of an inefficient material for the present invention is one that radiates at a suitable wavelength, but the amount of material needed to be effective is commercially and/or cosmetically infeasible.

Like radiance, the emissivity of a material also depends on the temperature of the material. Thus, in addition to radiance verses wavelength, one could look at emissivity verses wavelength curves to find materials in the 0.15 to 30 μm wavelength range that have high emissivity, in a temperature range of interest, i.e. 25° C.-175° C. or 40° C.-60° C. or 60° C.-80° C. and so forth. Suitable materials have emissivity greater than about 0.50. Preferred materials have an emissivity greater than about 0.80. Materials most preferred have emissivity greater than about 0.90.

Thus, initial requirements for a suitable material include: one that that has emissivity greater than about 0.50, so that when heated to 25° C.-175° C. the material emits in the 0.15 to 30 μm wavelength range, at an intensity that is useful to reshape human hair in a commercially acceptable amount of time.

c. Commercial Considerations

To be a suitable material, we must be able to use the material in quantities that are commercially reasonable for use in a cosmetic product, while still being effective. What is “commercially reasonable” depends on cost, manufacturing difficulties, ability to stabilize the composition, look, feel, smell and overall impression of the composition, etc. So, for example, if an otherwise useful material imparts a foul odor to the composition in which it is disposed, then that material is less suitable or not at all suitable. Or if an otherwise useful material destabilizes the composition in which it is disposed, then that material is less suitable or not at all suitable. A person of ordinary skill in the art is able to identify a composition of unacceptable consumer quality or low commercial viability, and is thus able to steer clear of materials that are not commercially reasonable.

Furthermore, a suitable material is one that is suitable for use in cosmetic preparations, from a safety standpoint; at a minimum meeting all relevant controlling regulations for cosmetic products. So, if an otherwise useful material is banned by all or some regulatory authorities, then that material is less suitable or not at all suitable, because a commercial product cannot be achieved. It was surprising that material(s) meeting all of the physical, formulary and commercial requirements herein discussed, could be found.

d. Activation/Deactivation of the Suitable Material

Furthermore, a preferred suitable material is one that must be activated before it will significantly affect the disulfide bonds in human hair, and which can be deactivated to stop the effect. Otherwise the hair would be subject to reshaping as soon the composition of the present invention was put on the hair (under the tensioning of gravity, for example) and would continue as long as some residue remained on the hair. It is realized that many materials, even at room temperature, emit some radiation in the 0.15 to 30 μm wavelength range. However, by “activated”, we mean that the intensity of radiation emitted by the suitable material is “useful to reshape human hair” in a “commercially acceptable amount of time”. Thus, if a suitable material is emitting radiation in the 0.15 to 30 μm wavelength range, but the intensity is such that reshaping hair requires more than one hour, then that material is not “activated” as herein defined.

Preferred methods of activation and deactivation must be suitable for consumer use and be commercially sensible in the personal care market. So, for example, if an otherwise useful material requires an activation/deactivation that is inconvenient from a consumer standpoint or that requires copious amounts of energy, then that material may not be suitable. A preferred activation method is heating with a hair dryer, either a handheld hair dryer or a commercial hair dryer typically found in hair salons. This method of activation is preferred, because it is already expected that compositions according to the present invention will be subjected to heat from a hair dryer or hair shaping tool, as the consumer goes about her usual grooming or beauty routine. Accordingly, a preferred suitable material is one that does not produce effective wavelengths and/or intensity, until the material is heated to 40° C. to 60° C., more preferably above 80° C., and most preferably between 60° C. and 80° C. A minimum of 40° C. is useful to prevent unwanted activation of the composition. Temperatures above 80° C. can be used to activate the suitable material, but the temperature itself begins to have a detrimental effect on the hair. Therefore, the most preferred activation temperatures are between about 60° C. and 80° C. These temperatures are achievable with a handheld hair dryer, even though the source of hot air is several inches from the hair and the hot air flow may not be continuously directed on the same portion of hair. Preferably, activation is achievable within ten minutes of blow drying, more preferably, within five minutes of blow drying, most preferably, within one minute of blow drying. We note that devices other than hair dryers may be used; for example flat irons. However, if a flat iron is used, it is preferably used to heat the suitable material to it's most preferred temperature, and no more, thereby limiting any damage from excessive heat.

We also expect that activation, depending on the emitting material, may be achievable by light. In this embodiment, shining a visible light (red, blue, green etc) on the suitable material causes the suitable material to radiate in the 0.15 to 30 μm wavelength range. The intensity of the emitted radiation, in general, depends on the intensity of the visible light activation source. But we expect that an effective and commercially viable combination of visible source light and suitable radiating material may be found. Deactivation is achieved by removing the visible light source. Activation and deactivation by this method would be essentially immediate, since there is no waiting for the suitable material to heat up.

e. Adjunct Treatments

Furthermore, although other hair shaping treatments could be combined with the principles of the present invention, a preferred suitable material and composition according to the invention is one that supplies enough electromagnetic radiation to reshape the hair to any desired shape, no adjunct treatments being needed.

### Tourmalines as Suitable Materials

Unexpectedly, we have noted that tourmalines are very useful in the compositions of the present invention. Referring to FIG. 1, this particular red tourmaline, heated to 78° C., has emissivity well over 0.9 in the wavelength range with which we are concerned. At 20 μm wavelength, the emissivity is about 0.93. Though not shown, the emissivity of this material, at 20 μm, drops to about 0.75 when the temperature is reduced to about 44° C.

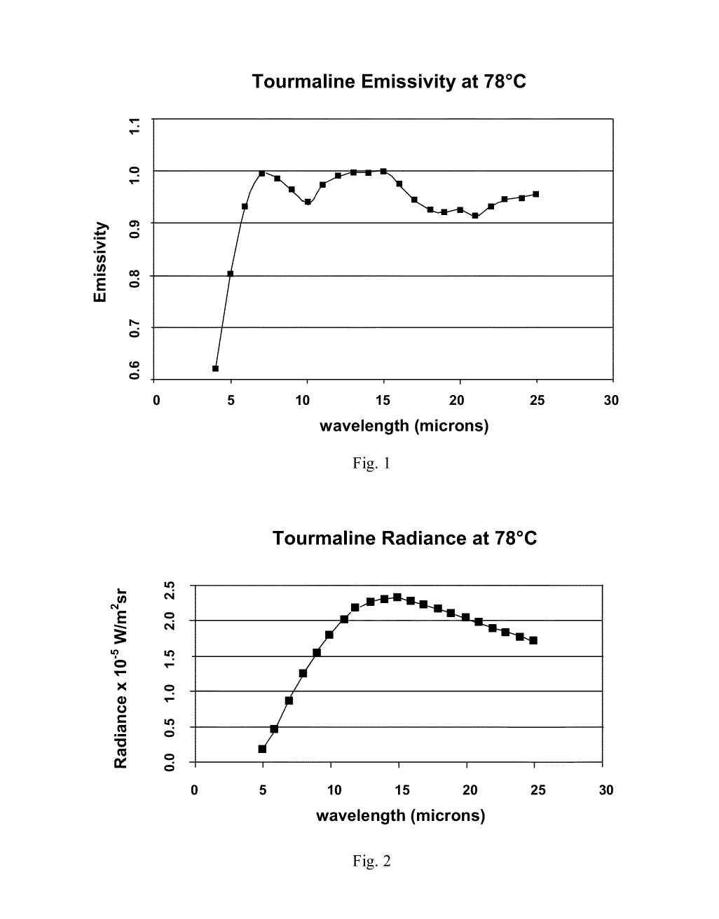

Referring to FIG. 2, the energy output of this particular red tourmaline, heated to 78° C., peaks between about 10 and 20 μm wavelength. 78° C. is a temperature that is not unusual for hair styling.

But having identified a material (the red tourmaline) with the right wavelength and high emissivity, the question remained, is the intensity sufficient to make a commercial product. In other words, what surface area of red tourmaline will emit enough energy to effectively reshape human hair in a commercially acceptable amount of time? Can that surface area be achieved in an amount by weight of tourmaline that can be incorporated into a commercially viable product?

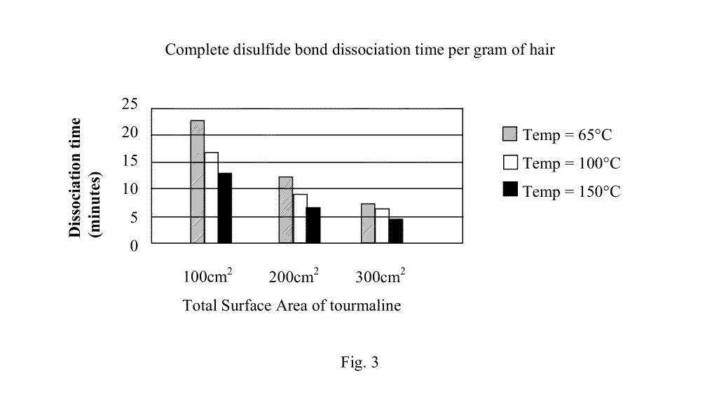

A full head of human hair may be on the order of 30 grams. It has been estimated that a single gram of human hair comprises on the order of 6×10²⁰ disulfide bonds. Given a dissociation energy of 3.52×10⁻¹⁹ joules per bond (2.2 eV), a single gram of hair requires about 211 joules to dissociate all the S—S bonds. Now, cosmetic hair reshaping does not require all of the disulfide bonds in the hair to be broken. Indeed, this would destroy the hair. Nevertheless, we calculated the time required for a black body (i.e. emissivity=1) to emit 211 joules of energy at different temperatures for given total surface area of the blackbody. The results are shown in FIG. 3. The graph indicates that, at temperatures achievable with ordinary blow dryers or flat irons, a commercially feasible surface area of high emissivity material could supply enough intensity to reshape the hair in an acceptable amount of time. We considered that tourmaline, with emissivity greater than 90% at working temperatures, is accurately modeled by these blackbody calculations.

Thus, it is demonstrated, for the first time, that permanent reshaping of human head hair, via disulfide bond reorganization, can be achieved by using a topical composition comprising red tourmaline, to directly affect the disulfide bonds. This treatment is considered non-chemical. By “non-chemical” we differentiate from known commercial treatments that break disulfide bonds through chemical interactions, rather than photon absorption. In using a composition of the present invention, no other type of treatment is necessary, nor preferred.

Surprisingly, tourmaline compositions that are safe, stable and commercially acceptable, as well as effective, were achieved. The tourmaline is used in quantities that are reasonable for commercial cosmetic products, and yet the tourmaline still supplies enough electromagnetic energy to effect a permanent reshaping of the hair, despite the fact that the emitted photon energy is well below the dissociation energy of a ground state disulfide bond. The tourmaline must be activated before it will significantly affect the disulfide bonds in human hair, and can be deactivated to stop the effect.

In another embodiment the activation of tourmaline is achieved by shining a visible light on the tourmaline. For example, we note that red and pink tourmalines have absorption lines at 458 and 451 nm, as well as a broad absorption band in the green spectrum. Blue and green tourmalines have a strong, narrow absorption band at 498 nm and almost complete absorption of red, down to 640 nm. In turn, these materials re-emit a portion of the incident light energy in the 0.15 to 30 μm wavelength range, and therefore, may be useful in effecting permanent hair reshaping. Suitable sources of visible light include LEDs and lasers. With these devices, the light can be concentrated and directed.

### Quantifying Performance of a Suitable Material and Composition

By the experimentation, now described, it is possible to determine if a material can reshape hair in a commercially acceptable amount of time. It is also possible to characterize many performance parameters of a composition containing a candidate suitable material.

One or more candidate suitable materials may be incorporated into a base composition. A standard section of hair can be identified according to any set of criteria that may be of interest. Criteria may include: mass of the section of hair, color, hair shape (straight or curly), ethnicity, condition (i.e. brittle or strong, limp or bouncy, dull or shiny, etc.) and so forth. Once a standard section of hair is defined, a defined amount of a composition containing a candidate material is applied to the standard section of hair and tension is applied. The tension is appropriate for the desired effect. For example, if the standard section of hair is curly, tensioning will straighten the hair. If the standard section of hair is straight, tensioning might include winding the hair around a curler. The tension will be maintained for a predetermined amount of time (i.e. five minutes or ten minutes or 30 minutes or one hour). During that time, the hair and composition will be heated to a predefined temperature. The temperature will be maintained for a predetermined length of time. It may be as long as tension is applied to the hair or shorter. After the heat and tension are removed, measurements can be made to quantify the degree of reshaping. Such measurements may be those well known in the art. In this manner, various comparisons may be made. For example, one candidate material may be compared to another; or one concentration of a candidate material may be compared to another concentration of the same candidate material; or one candidate in a particular base composition may be compared to the same candidate material in a different base composition; or a candidate material may be compared to itself when combined with an adjunct hair treatment (i.e. water rinse, shampoo, blow dry). Additionally, we may want to know what happens when the composition is removed from the hair shortly after tensioning is stopped, compared to what happens when the same composition is left in the hair for an extended period after tensioning is stopped. A keen observer may also note differences in the time that the section of hair hold the new shape, given the ambient humidity, temperature and light exposure.

### Proof of Concept

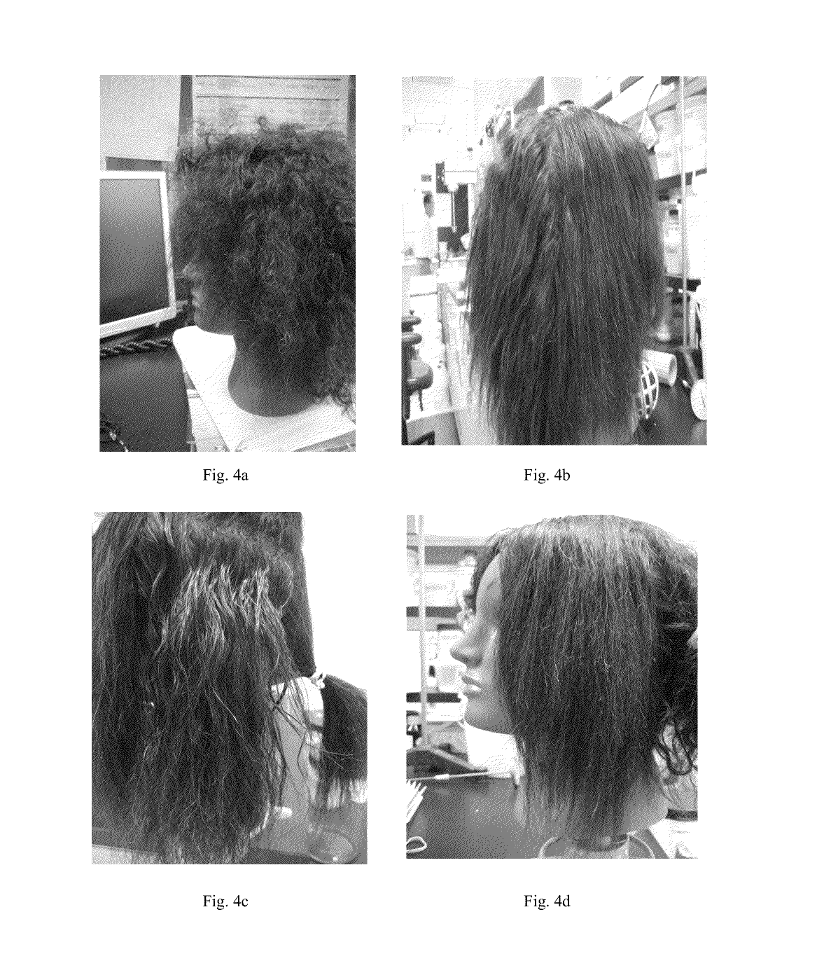

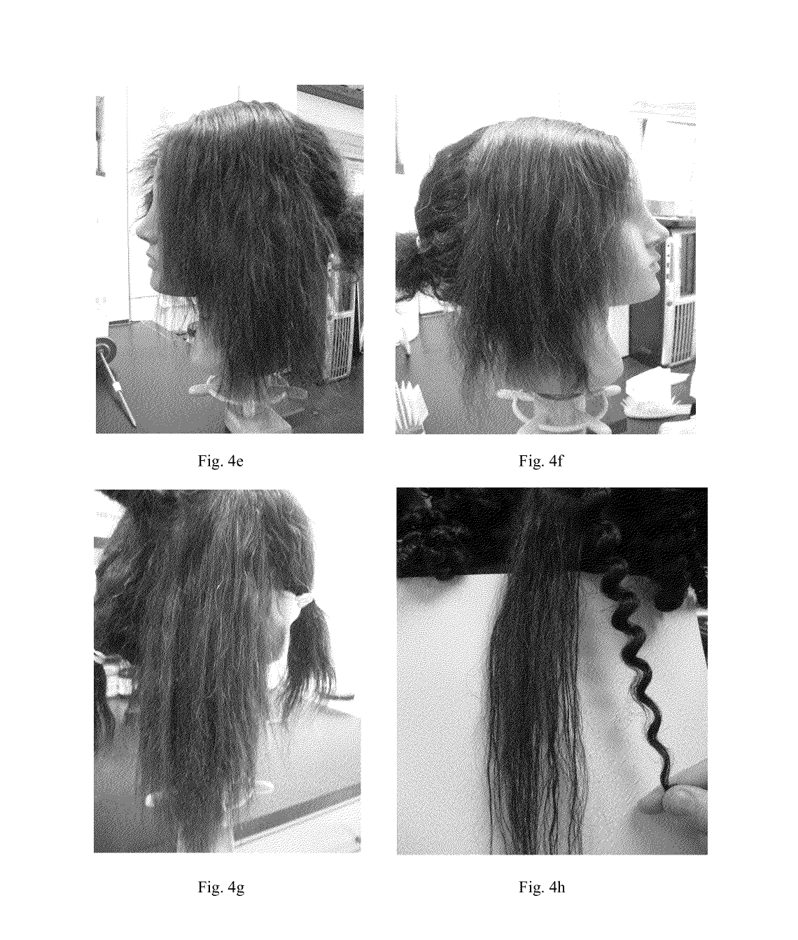

To demonstrate the concept of the present invention, we attempted to straighten naturally curly hair and permed hair. For this proof of concept test, 5% by weight of red tourmaline grind or powder was dispersed in a 2% solution of CARBOPOL® 98 (20%), shea butter (10%), lecithin (2%) and PVP (3%); the remainder, water. The formula with no tourmaline (a.k.a. base formula) served as a control. The creams were evenly applied to dry hair, from the root to the free end. The amount applied was typical for this type of hair treatment. The tensioning applied to the hair was that associated with blow drying and/or combing and incidental handling. No excessive tensioning was used. The following test samples were set up, and results can be seen in FIGS. 4a-4h.

FIG. 4a Control=ethnic hair, untreated.
FIG. 4b Negative Control=ethnic hair, washed with water, then blow dried.
FIG. 4c Negative Control=ethnic hair, treated with the base formula, then blow dried.
FIG. 4d Positive Control=ethnic hair, treated once with MIZANI® Rhelaxer, washed with water, then blow dried.
FIG. 4e Ethnic hair treated with 5% Red Tourmaline formula, then blow dried (activated).
FIG. 4f Ethnic hair treated with 5% Red Tourmaline formula, then blow dried (activated), then washed with hot water, then blow dried.
FIG. 4g Ethnic hair treated with 5% Red Tourmaline formula, then air dried (no activation), then shampooed with hot water (removing much of the tourmaline), then blow dried; then shampoo and blow dry were applied a total of five times in succession.
FIG. 4h (right side) Negative Control=Caucasian permed hair, washed with water, then blow dried; (left side) Caucasian permed hair treated with 5% Red Tourmaline formula, then blow dried.

In FIG. 4a, the ethnic control hair is quite curly. The left side of FIG. 4b shows that same hair after being washed and blow dried. FIG. 4c shows the hair after being treated with the base formula. While the straightening seems dramatic, it is not permanent because the disulfide bonds have not been altered. These hair samples will revert to their original shape in a short amount of time. FIG. 4d is the ethnic hair straightened with a commercial product (MIZANI® Rhelaxer). FIG. 4e shows the ethnic hair treated with 5% red tourmaline formula, and the tourmaline was activated by blow drying the hair. The straightening is comparable to the MIZANI® Rhelaxer in FIG. 4d. FIG. 4f shows the ethnic hair after being treated with the 5% red tourmaline formula, then activated by blow drying, then washed with hot water, then blow dried. It is clear, that subsequent washing did not undo the straightening effect, which is quite dramatic. FIG. 4 g shows the ethnic hair treated with 5% red tourmaline formula, then air dried, so little or no activation of the tourmaline has occurred, then shampooed with hot water (removing some amount of the tourmaline), then blow dried (activating the remaining tourmaline); then shampoo and blow dry were applied a total of five times in succession. A good measure of straightening was still achieved, but not as much as in FIGS. 4e and 4f However, it is again clear, that subsequent washing did not undo the straightening effect. Presumably, this is because some of the tourmaline was rinsed out of the hair before activation. FIG. 4h has two parts. On the right is Caucasian permed hair, washed with water, then blow dried. The hair is quite curly. On the left is the same type of hair after being treated with 5% red tourmaline formula and activated by blow drying. The straightening effect is dramatic. Clearly, the tourmaline composition and activation by blow drying are able to straighten hair that is either naturally curly or previously permed.

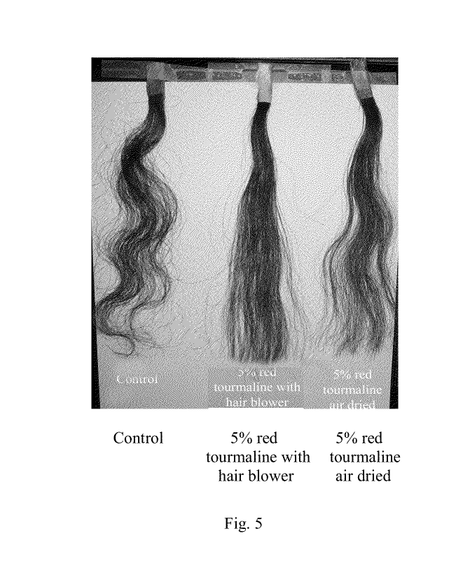

FIG. 5 shows the permanence of a straightening treatment and the improvement achieved when activated with a blow dryer. On the left is an untreated control sample of wavy hair. On the right is hair treated with 5% red tourmaline formula at ambient temperature (no hair dryer). In the center is hair treated with 5% red tourmaline formula and activated with hair dryer. The sample on the right is clearly straighter than the control sample. And the sample in the center is still better. Thus, elevating the temperature of the tourmaline definitely improves the result. Furthermore, FIG. 5 shows the test samples two months after treatment was applied. Thus, the treatment is considered permanent, as herein defined.

### Colorimetric Disulfide Bond Analysis

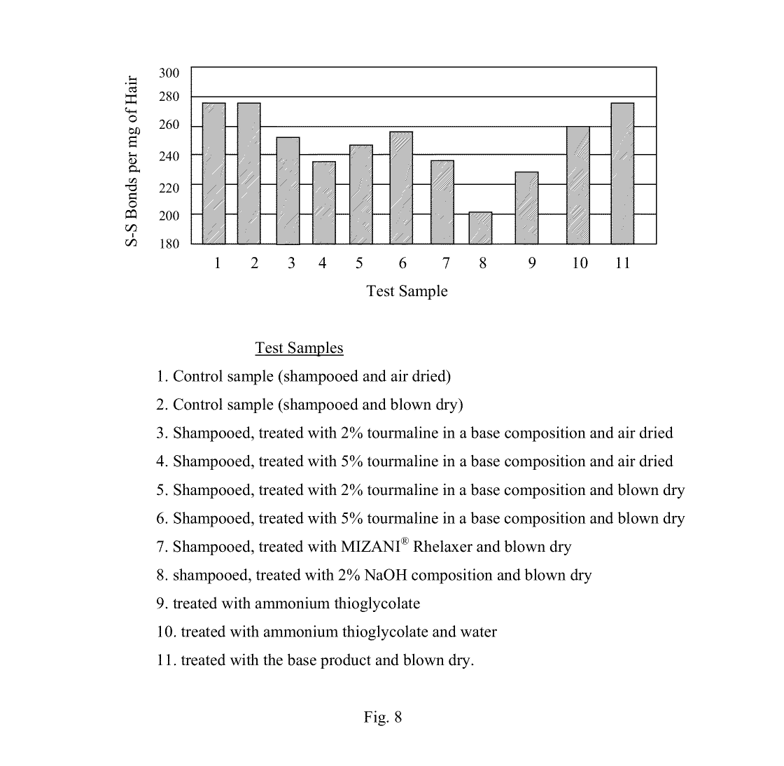

The hair cysteine content (disulfide bond content) was quantified by measuring the amount of oxidized dithiothreitol (DTT) (λmax=280 nm) derived from DTT with cysteine. The following test samples were prepared: 1. Control sample (shampooed and air dried); 2. Control sample (shampooed and blown dry); 3. Shampooed, treated with 2% tourmaline in a base composition and air dried; 4. Shampooed, treated with 5% tourmaline in a base composition and air dried; 5. Shampooed, treated with 2% tourmaline in a base composition and blown dry; 6. Shampooed, treated with 5% tourmaline in a base composition and blown dry; 7. Shampooed, treated with MIZANI® Rhelaxer and blown dry; 8. shampooed, treated with 2% NaOH composition and blown dry; 9. treated with ammonium thioglycolate; 10. treated with ammonium thioglycolate and water; 11. treated with the base product and blown dry. The results are depicted in FIG. 8. The results show a reduction of disulfide bonds for the hair treated with the tourmaline and for the hair treated with chemical treatments (i.e. MIZANI® Rhelaxer, NaOH, ammonium thioglycolate). Colorimetric disulfide bond measurements show that in this study, tourmaline reduced about 6-13% of S—S bonds in hair at acidic pH compared to control.

### Hair Damage

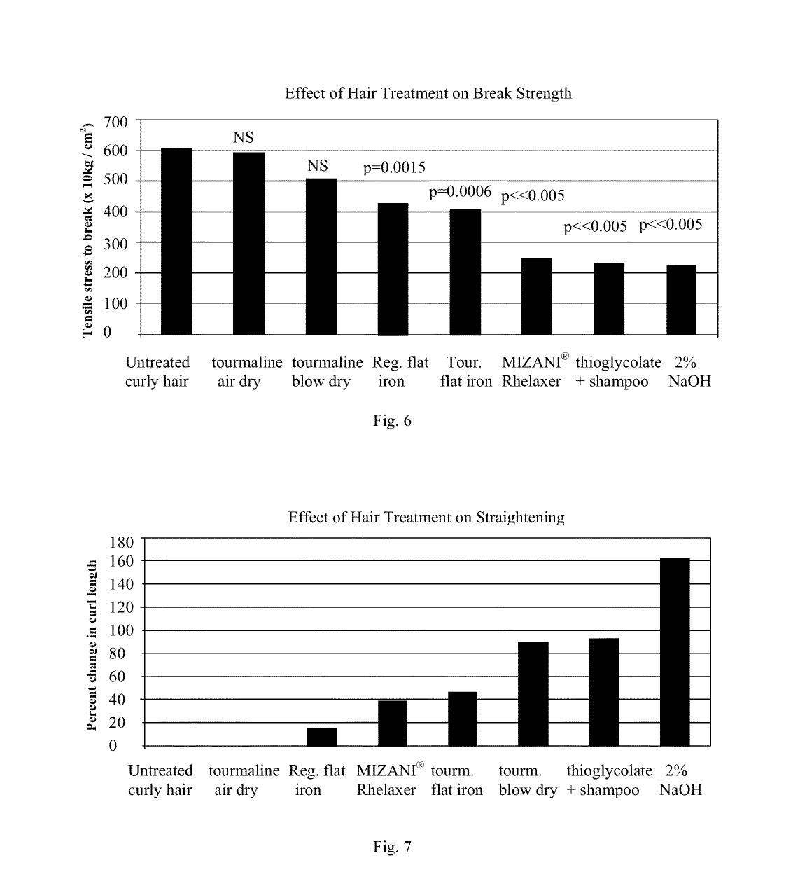

One of the main advantages of the present invention over chemical treatments, is the avoidance of damage associated with chemical treatments. To demonstrate that no significant damage is done to the hair when treated according to the present invention, the tensile stress at break was measured, before and after various types of straightening treatment. An Instron tensile test machine, Model 5542 with mini grips and 10N load cell, was used. The extension rate was 20 mm/minute. The samples were 4 mm lengths of Caucasian hair (curly before treatment, straightened after treatment). All samples were tested under the same conditions: the samples were dry, the ambient temperature and humidity were 70° F. and 20%, respectively. Reading left to right in FIG. 6, the samples are:
Negative control: untreated curly hair
Straightened with 5% red tourmaline formula described above, washed out and air dried
Straightened with the 5% red tourmaline formula, washed out and blow dried
Positive control: straightened with flat iron containing no tourmaline
Positive control: straightened with a tourmaline-containing flat iron
Positive control: straightened with a sodium hydroxide commercial product (MIZANI® Rhelaxer)
Positive control: straightened with thioglycolate commercial product, shampoo and rinse
Positive control: straightened with 2% sodium hydroxide aqueous solution, neutralized with citrate

Hair damage caused by treatment is manifest as a reduction in stress to break. The results in FIG. 6 indicate that hair straightened with the 5% red tourmaline composition, compared to the untreated curly hair, was not weakened to a statistically significant degree. This is unlike all of the other samples, especially those treated by conventional chemical methods.

### More Advanced Study of Tensile Properties of Hair

Four groups of sixty hair fibers each, were prepared as follows. One group was a control, washed in Bumble and Bumble Alojoba Shampoo, rinsed and blown dry; one group was treated with MIZANI® Rhelaxer, 0.3 mL applied to each fiber; one group was treated with 2% red tourmaline in a cream base (see formula 1), 0.2 mL applied to each fiber, followed by blow dryer heating for about 5 minutes; one group was treated with 2% red tourmaline in a gel base (see formula 2), 0.2 mL applied to each fiber, followed by blow dryer heating for about 5 minutes.

| | Percent |
|---|---|
| | by weight |
| Ingredients | of composition |
| purified water | 67.70 |
| Aristoflex ® AVC (Ammonium | 1.00 |
| Acrylodimethyltaurate/VP | |
| Copolymer) | |
| glycerine | 2.00 |
| phenoxyethanol | 0.70 |
| Polyvinylpyrrolidone (PVP) | 3.00 |
| glycerin/water/sodium PCA/ | 5.00 |
| urea/trehalose/polyquaternium- | |
| 51/sodium hyaluronate | |
| cetearyl alcohol/cetearyl glucoside | 4.60 |
| PEG-100 stearate | 1.00 |
| cetyl alcohol | 2.00 |
| petrolatum | 3.00 |
| shea butter | 5.00 |
| polyquaternium-7 | 2.50 |
| red tourmaline | 2.00 |
| caprylyl glycol/phenoxyethanol/ | 0.50 |
| hexylene glycol | |

### Formula 1-2% Red Tourmaline Cream

| | Percent by weight |
|---|---|
| Ingredients | of composition |
| purified water | 86.30 |
| carbomer | 1.00 |
| glycerine | 2.00 |
| phenoxyethanol | 0.70 |
| Polyvinylpyrrolidone (PVP) | 3.00 |
| glycerin/water/sodium PCA/ | 5.00 |
| urea/trehalose/polyquaternium- | |
| 51/sodium hyaluronate | |
| red tourmaline | 2.00 |

### Formula 2-2% Red Tourmaline Gel

The fibers were then equilibrated to 80% relative humidity and run to break on the Diastron MTT675 automated tensile testing machine. Results of the tensile testing are shown in the following table.

| | | |
|---|---|---|
| Mean | SD | Significance |

Young's modulus

| | | | |
|---|---|---|---|
| control | 1.87E+09 | 2.24E+08 | |
| MIZANI ® | 1.44E+09 | 2.6E+08 | p < 0.001 |
| Rhelaxer | | | |
| 2% tourmaline | 1.62E+09 | 2.99E+08 | p < 0.001 |
| gel | | | |
| 2% tourmaline | 1.99E+09 | 2.19E+08 | p < 0.02 |
| cream | | | |

Stress at 15%

| | | | |
|---|---|---|---|
| control | 8.01E−03 | 3.28E−04 | |
| MIZANI ® | 6.74E−03 | 5.75E−04 | p < 0.001 |
| Rhelaxer | | | |
| 2% tourmaline | 7.40E−03 | 3.67E−04 | p < 0.001 |
| gel | | | |
| 2% tourmaline | 7.49E−03 | 8.48E−04 | p < 0.001 |
| cream | | | |

Work at 15%

| | | | |
|---|---|---|---|
| control | 1.67E−03 | 2.82E−04 | |
| MIZANI ® | 1.38E−03 | 2.34E−04 | p < 0.001 |
| Rhelaxer | | | |
| 2% tourmaline | 1.38E−03 | 2.15E−04 | p < 0.001 |
| gel | | | |
| 2% tourmaline | 1.48E−03 | 2.72E−04 | p < 0.001 |
| cream | | | |

Post Yield Gradient

| | | | |
|---|---|---|---|
| control | 1.23E−03 | 1.49E−04 | |
| MIZANI ® | 1.26E−03 | 1.71E−04 | NS |
| Rhelaxer | | | |
| 2% tourmaline | 1.28E−03 | 1.60E−04 | NS |
| gel | | | |
| 2% tourmaline | 8.76E−04 | 1.37E−04 | p < 0.001 |
| cream | | | |

Break Extension

| | | | |
|---|---|---|---|
| control | 42.74 | 6.16 | |
| MIZANI ® | 45.72 | 6.55 | p < 0.02 |
| Rhelaxer | | | |
| 2% tourmaline | 43.65 | 7.16 | NS |
| gel | | | |
| 2% tourmaline | 44.38 | 5.85 | NS |
| cream | | | |

Break Stress

| | | | |
|---|---|---|---|
| control | 1.72E−02 | 2.87E−03 | |
| MIZANI ® | 1.73E−02 | 3.11E−03 | NS |
| Rhelaxer | | | |
| 2% tourmaline | 1.73E−02 | 3.34E−03 | NS |
| gel | | | |
| 2% tourmaline | 1.45E−02 | 1.95E−03 | p < 0.001 |
| cream | | | |

Total Work

| | | | |
|---|---|---|---|
| control | 7.13E−03 | 2.08E−03 | |
| MIZANI ® | 7.16E−03 | 1.82E−03 | NS |
| Rhelaxer | | | |
| 2% tourmaline | 6.60E−03 | 2.16E−03 | NS |
| gel | | | |
| 2% tourmaline | 6.25E−03 | 1.50E−03 | p < 0.01 |
| cream | | | |

In the study of hair, changes in the linear (Hookian) and yield regions are normally associated with changes in the moisture content of the fiber, and the ability of the keratin to maintain it's helical form. The covalent parameters (break stress, work done to break, and post yield extension) give a measure of the covalent (molecular) properties of the fiber, which give an indication of fiber strength and fiber damage.

Treatments with both 2% tourmaline compositions had a highly significant effect on the Hookian and yield regions of the stress-strain curve. Young's Modulus, stress at 15% strain and Work Done at 15% strain, were all reduced. This may be interpreted as the tourmaline treatments plasticizing the hair, making it more compliant. A reduced Young's modulus means softer, less brittle hair. On the other hand, the covalent parameters were largely unaffected by these two tourmaline treatments (except for a small increase in break extension associated with the 2% tourmaline gel treatment). Thus, the hair fibers do not seem to have sustained any damage.

In contrast, the MIZANI® Rhelaxer treatment caused a significant increase in Young's Modulus, while reducing the yield parameters (stress at 15% strain and Work Done at 15% strain). The increase in Young's modulus implies that the fibers became more brittle as a result of treatment. Furthermore, this treatment significantly reduced the covalent parameters (break stress, work done to break, and post yield extension), clearly indicating a weakening of the fiber's protein structure due to molecular damage.

### Comparison of Hair Straightening Methods

To demonstrate the effectiveness of compositions according to the present invention, the percent straightening of curly hair was measured, after various types of treatment. Curl length after treatment was measured and compared to untreated curly hair and expressed as a percent change. All samples were measured under the same conditions: the samples were dry, the ambient temperature and humidity were 70° F. and 20%, respectively. Reading left to right in FIG. 7, the samples are:
Negative control: untreated curly hair
Straightened with 5% red tourmaline formula described above, washed out and air dried
Positive control: straightened with flat iron containing no tourmaline
Positive control: straightened with MIZANI® Rhelaxer
Positive control: straightened with a tourmaline-containing flat iron
Straightened with 5% red tourmaline composition, washed out and blow dried
Positive control: straightened with thioglycolate commercial product, shampoo and rinse
Positive control: straightened with 2% sodium hydroxide aqueous solution, neutralized with citrate

The results in FIG. 7 indicate that the hair straightened with 5% red tourmaline composition that has been heated with a blow dryer straightens hair better than or at least as well as the commercial chemical straighteners or the flat irons. We note that the 5% tourmaline composition plus air drying was ineffective. Thus, heating the tourmaline composition to activation was crucial in this test. Interestingly however, the 5% tourmaline grind composition with blow dry performed twice as well as the tourmaline-containing flat iron. This indicates that the tourmaline embedded in the flat iron, is not having the same effect as the 5% tourmaline composition applied to the hair, even though the tourmaline flat iron achieves much higher temperatures than the blow dryer used with the 5% composition. Thus, we conclude that a tourmaline-containing flat iron does not perform at all like the tourmaline composition of the present invention. The tourmaline flat iron does not read on compositions or methods of the present invention.

### Effect of Tourmaline on Secondary Protein Structure: X-Ray Scattering

Wide angle x-ray scattering (WAXS) and small angle x-ray scattering (SAXS) were used to determine protein keratin structure of hair fibers after various treatments.

Five samples were prepared. A control sample was not treated; one sample was treated with a base formula and blown dry; one sample was treated with 2% tourmaline in the base formula and blown dry; one sample was treated with 2% NaOH (pH=13.45) and blown dry; one sample was treated with 4% urea in the base formula (pH=7) and blown dry.

The WAXS data provides information about secondary protein keratin structures such as: alpha, beta, alpha+beta, etc. of the hair fiber, where as the SAXS data provides information about longitudinal distance structure in the hair fiber between 1-100 nm such as coiled-coil, amorphous, ordered glycoprotein molecules, etc. For the analysis that follows, “strong” is defined as a dominant/sharp protein structure of the sample, “weak” is defined as existing/broad/vague protein structure, and “absent” means the structure is not present in the hair fiber. “Appearing” is defined as the space where the x-ray measured the protein structure. In 2-dimensional x-ray scattering images, we were able to clearly distinguish strong, weak and absent protein structures. These show up as strong, weak or absent reflections (arc/dot/ring), at certain values of the scattering vector q. The results, including q values, are listed in the next two tables.

| | | | .98 nm* |
|---|---|---|---|
| | .51 nm* | .465 nm* | Alpha & beta |
| Treatment | Alpha keratin | Beta keratin | keratin |
| Control | Strong, appear at | Weak, appear at | Weak, appear at |
| | 0.58 nm | 0.51 nm | 1.20 nm |
| | (q = 1.08 A⁻¹) | (q = 1.23 A⁻¹) | (q = 0.52 A⁻¹) |
| Base formula | Strong, appear at | Absent | Absent |
| | 0.58 nm | | |
| | (q = 1.08 A⁻¹) | | |
| 2% Tourmaline | Strong, appear at | Strong, appear at | Strong, appear at |
| | 0.56 nm | 0.48 nm | 0.97 nm |
| | (q = 1.12 A⁻¹) | (q = 1.31 A⁻¹) | (q = 0.64 A⁻¹) |
| 2% NaOH | Absent | Weak, appear at | Weak, appear at |
| | | 0.52 nm | 1.16 nm |
| | | (q = 1.21 A⁻¹) | (q = 0.54 A⁻¹) |
| 4% Urea | Strong, appear at | Strong, appear at | Weak, appear at |
| | 0.57 nm | 0.48 nm | 1.20 nm |
| | (q = 1.10 A⁻¹) | (q = 1.31 A⁻¹) | (q = 0.52 A⁻¹) |

WAXS Data: q = x(A⁻¹), scattering vector = 4πsinθ/λ, where λ is x-ray wavelength and scattering angle 2θ.

| | Meridional | | | |
|---|---|---|---|---|
| | reflection | Reflection | Reflection | Reflection |
| Treatment | 6.7 nm^{(a)} | 5.8 nm | 4.65 nm^{(b)} | 4.0 nm |
| Base formula | Absent | Weak | Absent | Strong, appear at |
| | | | | 4.0 nm |
| | | | | (q = 1.57 nm⁻¹) |
| 2% Tourmaline | Strong, appear at | Strong, appear at | Weak, appear at | Strong, appear at |
| | 6.7 nm | 5.8 nm | 4.7 nm | 4.0 nm |
| | (q = 0.94 nm⁻¹) | (q = 1.08 nm⁻¹) | (q = 1.34 nm⁻¹) | (q = 1.57 nm⁻¹) |
| 2% NaOH | Absent | Absent | Absent | Absent |
| 4% Urea | Strong, appear at | Weak, appear at | Strong, appear at | Weak,appear at |
| | 6.7 | 5.8 nm | 4.7 nm | 4.0 nm |
| | (q = 0.94 nm⁻¹) | (q = 1.08 nm⁻¹) | (q = 1.34 nm⁻¹) | (q = 1.57 nm⁻¹) |
| Control | Weak, appear at | Absent | Absent | Weak, appear at |
| | 6.7 nm | | | 4.0 nm |
| | (q = 0.94 nm⁻¹) | | | (q = 1.57 nm⁻¹) |

SAXS Data: q = x(nm⁻¹), scattering vector = 4πsinθ/λ, where λ is x-ray wavelength and scattering angle 2θ.
Notes:
^{(a)}6.7 nm sharp meridional reflection is determined by the coiled-coil keratin structure; 6.7 is the seventh order of the main period.
^{(b)}4.65 nm reflection is interpreted as arising from the order of the glycoprotein moleculars ordered in the liquid-crystalline structure, which is related to the flexible ECM.

The control sample has a strong alpha keratin structure, weak beta structure, and a weak alpha+beta structure. It has a coiled-coil structure. It should be noted that WAXS shows a very weak peak at 0.40 nm for this sample, however its protein structure is not identified, and could be due to non-homogeneous hair structure. The SAXS data shows that the control sample has the 6.7 nm meridional reflection, which corresponds to the coil-coil keratin structure.

The base formula sample has a strong alpha keratin structure, with two signature features in the wide angle x-ray scattering region: (1) a broad equatorial spot centered at 1.15 nm, corresponding to the mean distance, or spacing, between alpha helical axes, and (2) a fine meridian arc at 0.58 nm, which is related to the projection of the alpha helical pitch along the coiled-coil axis, above a broader arc around 0.57 nm of less ordered coiled coil. The sample has no coiled-coil structure and no ordered glycoprotein moleculars. The SAXS data shows structures at 5.8 nm and 4.0 nm for sample 0, however their shapes are not identified. This could be due to non-homogeneous hair structure.

The WAXS data shows that the 2% tourmaline sample has a strong alpha structure, a strong beta structure, and a strong alpha+beta structure. The SAXS data points to both coiled-coil structure and ordered glycoprotein moleculars.

The 2% NaOH sample is different from the others. It has no alpha keratin structure, no meridian arc around 1.58 nm, and no equatorial spots. A weak beta structure (equatorial arc at 0.52 nm), and a weak alpha+beta structure are detected. It should be noted that WAXS shows a very weak peak at 0.36 nm for Sample 2, however its protein structure is not identified, and could be due to non-homogeneous hair structure. The SAXS data shows that sample 2 has no ordered molecular protein structure.

The 4% urea sample has a strong alpha structure, a strong beta structure, and a weak alpha+beta structure. It has both coiled-coil structure and a 4.7 nm peak (ordered glycoprotein moleculars) that is more pronounced than that of the 2% tourmaline sample.

Based on the WAXS data, the effect of the 2% tourmaline treatment seems to be the development of a strong beta structure and a strong alpha+beta structure.

Based on the SAXS data, the effect of the 2% tourmaline treatment seems to be the development of coiled-coil structure and ordered glycoprotein moleculars.

It may seem unlikely that the formation of new secondary structure could not be accounted by the breaking and/or rearranging of disulfide bonds in the hair, because disulfide interactions are a tertiary structure. But this may not be the case. For example, it may be only after a sufficient number of disulfide bonds are broken, that some other agent is able to influence the secondary structure. At any rate, we have definitively observed that activated tourmaline cleaves disulfide bonds, enhances secondary structure, and permits a permanent reshaping of hair, by non-chemical means. Not wishing to be bound by any on theory, the development of additional secondary structure, may well explain at least some of the permanent nature of the hair reshaping, observed above. The rearrangement of di-sulfide bonds and added secondary structure may be working together to hold the hair in a new shape.

### Other Suitable Materials

Only following these proof of concept tests, has it become clear that materials other than the red tourmaline are likely to be useful in the present invention. For example, various other tourmalines (i.e. black, green, pink, brown, blue) are expected to be similarly useful as red tourmaline. Also useful may be various ceramics and non-metals that emit radiation in the near and middle infrared, and that have emissivities above 90% at the working temperatures described herein. Graphite, gypsum and clays may be examples of useful non-metals. Any candidate material must satisfy the criteria discussed above.

### Compositions

Compositions of the present invention must satisfy certain criteria. For example, the compositions must be cosmetically acceptable and commercially viable. “Cosmetically acceptable” and commercially viable” or the like, usually imply that a composition is stable under typical conditions of manufacture, distribution and consumer use. By “stable”, we mean that one or more characteristics of a personal care composition do not deteriorate to an unacceptable level within some minimum period of time after manufacture. Preferably, that minimum time is six months from manufacture, more preferably one year from manufacture, and most preferably more than two years from manufacture.

Compositions of the present invention must be efficacious when used in reasonable amounts. A composition is considered effective to permanently reshape human hair, only if the amount of composition applied to the hair is what a consumer would consider reasonable. For example, if a lotion composition reshapes the hair, but a gallon of the composition is required, then this is not an effective composition according to the present invention. A person skilled in the art of personal care hair products has a very good idea of what consumers would consider reasonable. The amount of a composition of the present invention required for one treatment depends on the type and amount of hair being treated and on the desired effect. However, experience suggests that preferably, about 5 ounces or less of a composition according to the present invention is effective to complete a treatment of a full head of hair; more preferably, about 2.0 ounces or less; most preferably, about 1.0 ounce or less. While these amounts are preferred for commercial and consumer reasons, the present invention also contemplates larger amounts, as the case may necessitate.

Within the guidelines, herein discussed, virtually any cosmetically acceptable or commercially viable composition, that is beneficial or benign to human hair, can serve as a base composition. Generally, one could say that the base composition should not absorb too much of the radiation emitted by the suitable material, and the base composition should not interfere with activation or deactivation of the suitable material. With those restrictions, a composition according to the present invention may contain any ingredients that are known to provide a benefit to the hair, any ingredients required to render a stable product, and any ingredients that render the product more cosmetically acceptable or commercially viable. For example, polyvinylpyrrolidone-based film formers are common hair product ingredients. In compositions according to the present invention, these or other film formers may help to maintain the tension in the hair while the disulfide bond reorganization is occurring. However, no film former is needed nor is it be integral to achieving the permanent reshaping effects discussed herein.

Compositions according to the present invention may contain chemical perming agents as an adjunct to the non-chemical mechanism disclosed herein. Preferably, however, a composition according to the present invention has no chemical agent or reagent that reacts with disulfide bonds. Preferably, the only mechanism of disulfide bond cleavage is direct excitation by electromagnetic radiation supplied from the suitable material in the composition.

Compositions according to the present invention may advantageously contain hair coloring agents. Hair coloring reactions of the type well known in the art, and disulfide bond cleavage as described herein, may exhibit synergistic effects.

The composition may have virtually any form, even solid or semi-solid, provided the composition can be distributed throughout the section of hair being treated, and along its length, from root to tip.

The suitable material may be added to the base composition or added during the manufacture of the base composition in any manner that the circumstances may require or allow. Some suitable materials may be incorporated into the composition by simple mixing, others may require pretreatments. The composition may be a mixture, a suspension, emulsion, a solid, a liquid, an aerosol, a gel, or mousse, just to name a few. The composition may be in the form of shampoo or conditioner. The composition may be hydrous or substantially anhydrous. “Substantially anhydrous” means less than about 10% total water content.

Tourmalines are expected to be useful at concentrations as low as about 1%. Regarding upper limits, in general, there may practical upper limits to the concentration of suitable material. After all, only so many disulfide bonds need to be reorganized to achieve a particular hair style. However, the practical upper limit of any particular suitable material depends on many factors, not the least of which is how much product does a consumer apply, expecting to get a certain result. Thus, in a commercial product, trial and error or consumer use testing may be the best way to determine the concentration of the suitable material. An example of a controlled trial and error experiment might be, styling hair samples with a defined amount of compositions comprising increasing concentrations of a suitable material, and observing the concentration at which no additional benefit is derived. The defined amount should be based on market knowledge of how much product consumers are likely to use for the given amount and type of hair. Useful compositions will contain up to about 1% of one or more tourmalines, preferably up to about 2% of one or more tourmalines, and more preferably up to about 5% of one or more tourmalines. Tourmalines are expected to be useful at concentrations up to at least about 10% of the composition, but a diminishing returns effect may result thereafter, depending on the exact nature of the composition, the temperature, the amount of hair being styled, the amount of product applied, etc. Other, more efficient emitter materials (higher emissivity) may be useful at concentrations well below 1%, while less efficient materials (lower emissivity) may only be useful at higher concentrations; above about 5% for example, or even above about 10%, for example.

Table 1 is an example of a cosmetically acceptable, commercially viable, effective composition according to the present invention, containing 5% tourmaline.

TABLE 1
5% Red Tourmaline Cream

| | Percent by |
|---|---|
| | weight of |
| Ingredients | composition |
| purified water | 65.20 |
| Aristoflex ® AVC (Ammonium | 1.00 |
| Acrylodimethyltaurate/VP | |
| Copolymer) | |
| glycerine | 2.00 |
| phenoxyethanol | 0.70 |
| Polyvinylpyrrolidone (PVP) | 3.00 |
| ceteryl alcohol | 4.60 |
| PEG-100 stearate | 1.00 |
| cetyl alcohol | 2.00 |
| petrolatum | 3.00 |
| shea butter | 5.00 |
| polyquaternium-7 | 2.50 |
| red tourmaline | 5.00 |
| glycerin/water/sodium PCA/ | 5.00 |
| urea/trehalose/polyquaternium- | |
| 51/sodium hyaluronate | |

### Methods

The present invention includes methods of using the compositions, herein described. A basic method includes providing a composition according to the present invention; activating the composition to emit the photons; and causing the photons to be directly absorbed by the disulfide bonds in the hair. More detailed methods include applying to a section of hair, a portion of a composition according to the present invention; applying tension to the section of hair to assume a desired shape; activating the portion of composition; deactivating the portion of composition; and releasing the applied tension. The portion of composition is preferably about 5 ounces or less, more preferably about 2 ounces or less, and most preferably about one ounce or less. The step of applying the composition includes distributing the composition throughout the section of hair being treated, and along its length, from root to tip. The step of activation may include directing a flow of hot air at the section of hair for a time sufficient to activate the composition and/or reshape the hair. Alternatively, the step of activation may include irradiating the section of hair with visible light, as from an LED or laser. Methods may include washing the hair before or after treatment. Methods may include repeating application to the same section of hair or using an adjunct treatment on the same section of hair.

The idea of a commercially viable, topically applied, safe and stable composition that reshapes hair via radiation is new and non-obvious. The results achieved were unexpected and unlike anything in the prior art. The hair is not subjected to harsh chemicals and no mal-odor occurs. Novelty and non-obviousness are partly demonstrated by the following facts: this is the first time that this problem has been identified; this description is the first disclosure of a list of criteria that a solution to the problem must satisfy; this is the first time that a composition that meets those criteria has been disclosed. In other words, we identified the problem, found some solutions, and also defined criteria for all other solutions to the problem.

What is claimed is:

1. A method of reshaping human hair comprising the steps of:
applying to a section of hair a portion of a topical hair-reshaping composition that is able to emit photons at an intensity and range of wavelengths that are effective to alter tertiary and or secondary protein structures in the hair; wherein the energy of the photons is less than the dissociation energy of a ground state disulfide bond; and wherein the range of wavelengths is between 0.15 and 30 μm;
heating the portion of composition to at least 60° C. to emit the photons; and
causing the photons to be directly absorbed by the disulfide bonds in the hair.

2. The method of claim 1 wherein the portion of composition is about 2 ounces or less.

3. The method of claim 1 wherein the step of heating includes directing a flow of hot air at the section of hair for a time sufficient to activate the composition and alter tertiary and/or secondary protein structures in the hair.

4. The method of claim 3 wherein the flow of hot air is supplied by a hair dryer.

5. The method of claim 1 wherein the composition comprises tourmaline.

6. The method of claim 5 wherein the composition comprises 1% to 10% tourmaline, by weight of the composition.

7. The method of claim 1 wherein the composition breaks disulfide bonds in hair by photon absorption, and not by chemical interaction.

8. A method of permanently reshaping human hair comprising the steps of:
applying to a section of hair a portion of a topical hair-reshaping composition that is able to emit photons at an intensity and range of wavelengths that are effective to alter tertiary and or secondary protein structures in the hair, wherein the composition comprises a material that has an emissivity of at least 0.80, in the 0.15 and 30 μm wavelength range, when the material is heated to at least 60° C.;
applying tension to the section of hair to assume a desired shape;
heating the portion of composition to at least 60° C. to emit the photons;
deactivating the portion of composition; and
releasing the applied tension.

9. The method of claim 8 wherein the portion of composition is about 2 ounces or less.

10. The method of claim 8 wherein the step of heating includes directing a flow of hot air at the section of hair for a time sufficient to activate the composition and alter tertiary and/or secondary protein structures in the hair.

11. The method of claim 10 wherein the flow of hot air is supplied by a hair dryer.

12. The method of claim 8 wherein the steps between and including applying tension and releasing tension are completed in less than about 30 minutes.

13. The method of claim 8 wherein the material is a tourmaline.

14. The method of claim 13 wherein the composition comprises 1% to 10% tourmaline, by weight of the composition.

15. The method of claim 8 wherein the composition breaks disulfide bonds in hair by photon absorption, and not by chemical interaction.
